(19) United States
(12) Patent Application Publication
(10) Pub. No.: US 2026/0345908 A1
(43) Pub. Date: Nov. 5, 2026

(54) GAP CREATION DEVICE

(71) Applicant: Owen Mumford Limited, Woodstock (GB)

(72) Inventors: Matthew John Dobson, Woodstock (GB); Rosie Hutt, Woodstock (GB)

(73) Assignee: Owen Mumford Limited, Woodstock (GB)

(21) Appl. No.: 19/737,415

(22) Filed: Jul. 10, 2026

Related U.S. Application Data
(63) Continuation of application No. 17/786,346, filed on Dec. 18, 2020.

(30) Foreign Application Priority Data
Dec. 20, 2019 (GB) 1919034.7

(51) Int. Cl. A61M 5/20; A61M 5/32

(52) U.S. Cl. CPC A61M 5/2033; A61M 5/326; A61M 2005/2073

(57) ABSTRACT

A plunger assembly (100) for use with an auto-injector and comprising: a cartridge (102); and a firing assembly (103) comprising a plunger rod (104) and a drive portion (105), the firing assembly (103) being axially moveable relative to the cartridge (102), wherein the firing assembly (103) is configured to couple to the cartridge (102) at a coupling point along a length of the cartridge (102) to fix an axial position of the drive portion (105) relative to the cartridge (102), and wherein the length of the cartridge (102) is configured to be extended in a rearward direction such that the coupling point and a forward end (106) of the plunger rod (104) move rearwards.

### RELATED APPLICATION(S)

This application is a continuation application of U.S. application Ser. No. 17/786,346, filed Jun. 16, 2022, which is a U.S. National Stage entry of PCT International Application No. PCT/EP/2020/086938, filed Dec. 18, 2020, which claims the benefit of and priority to UK Patent Application No. 1919034.7, filed Dec. 20, 2019, the contents of each of which are incorporated herein by reference in their entireties.

### TECHNICAL FIELD

The invention relates to auto-injectors and, more specifically, to plunger assemblies for use with auto-injectors and methods of assembling auto-injector subassemblies.

### BACKGROUND

An auto-injector is a device for receiving a syringe and for driving a syringe plunger into a barrel of the syringe, typically for drug delivery, without any force being applied by the user. Typically, an auto-injector includes a plunger driver, often comprising a spring that is arranged to provide a force to drive the syringe plunger into the barrel. The plunger driver is typically activated by operation of a release mechanism on the auto-injector, such as a button or through pressure applied to an injection site via the auto-injector. A safety auto-injector may be one which includes a shroud that may be deployed to a position covering a needle of a syringe received within the auto-injector after use of the auto injector. The shroud of the auto-injector may be deployed under a force applied by a driver, which may be the plunger driver or a separate shroud driver.

Auto-injectors may be provided to a user containing a syringe which is pre-filled with a substance (such as a drug) such that the auto-injector is ready to use. Auto-injectors may therefore be transported to a user with the substance already contained within a syringe within the auto-injector. The fill volume of the pre-filled syringe may vary depending on the dose of medicament that is to be dispensed to the user.

### SUMMARY

According to the invention in a first aspect, there is provided a plunger assembly for use with an auto-injector, the plunger assembly comprising a cartridge; and a firing assembly comprising a plunger rod and a drive portion, the firing assembly being axially moveable relative to the cartridge, wherein the firing assembly is configured to couple to the cartridge at a coupling point along a length of the cartridge to fix an axial position of the drive portion relative to the cartridge, and wherein the length of the cartridge is configured to be extended in a rearward direction such that the coupling point and a forward end of the plunger rod move rearwards.

Optionally, the firing assembly is telescopically received within the cartridge.

Optionally, the plunger assembly further comprises firing assembly locking features configured to engage with cartridge locking features for coupling the firing assembly to the cartridge.

Optionally, the firing assembly locking features are configured to engage with the cartridge locking features under relative rotation between the firing assembly and the cartridge.

Optionally, the cartridge comprises a first portion and a second portion, axially moveable relative to the first portion for extending the length of the cartridge rearwards.

Optionally, the first portion is forward of the second portion, and wherein the firing assembly is configured to couple to the second portion.

Optionally, one of the first and second portions of the cartridge comprises an angled surface configured to interact with a cartridge follower on the other of the first and second portions of the cartridge and configured to ride over the angled surface, and extending the cartridge rearwards comprises rotating the second portion relative to the first portion to cause the corresponding feature to ride over the angled surface.

Optionally, the first portion further comprises a rotation lock configured to rotationally fix the first portion relative to a housing of the auto-injector.

Optionally, the plunger assembly further comprises an actuator axially coupled to the cartridge such that an axial force applied to the actuator inserts the cartridge into the auto injector, wherein the axial coupling is configured to be overcome on continued application of the axial force when forward movement of the cartridge is prevented, and wherein the actuator further comprises a cartridge coupler configured to couple the firing assembly to the cartridge on the continued application of the axial force.

Optionally, one of the actuator and cartridge comprises an angled surface that is configured to interact with a locking follower on the other of the actuator or the cartridge for translating, at least partially, the continued application of the axial force into a rotational force, and wherein the angled surface and the locking follower are configured to provide the axial coupling.

Optionally, the actuator comprises a sleeve configured to receive the cartridge therein.

Optionally, the, or a further, ramped surface is configured to interact with the locking follower, or a further follower, for rotating the second portion of the cartridge relative to the first portion of the cartridge.

According to the invention in a further aspect, there is provided an auto-injector subassembly comprising a plunger assembly according to any of claims 1 to 13, and further comprising a housing configured to receive the plunger assembly.

Optionally, the auto-injector subassembly comprises rotational engagement features configured to engage the rotational lock.

Optionally, the auto-injector subassembly comprises an axial end stop configured to receive the plunger assembly and to prevent further forward movement of the plunger assembly.

Optionally, the housing is configured to receive a syringe, and further configured to position the syringe such that at least part of the syringe forms the axial end stop.

According to the invention in a further aspect, there is provided an auto-injector subassembly comprising a plunger assembly and a housing, the plunger assembly comprising: a cartridge insertable within the housing; and a firing assembly comprising a plunger rod and a drive portion, the firing assembly being axially moveable relative to the cartridge, wherein the firing assembly is configured to couple to the cartridge at a coupling point along a length of the cartridge to fix an axial position of the drive portion relative to the cartridge, wherein the cartridge is configured to couple to the housing at a point during insertion therein, and wherein at least part of the cartridge is configured to be moveable rearwards to cause rearwards movement of the coupling point and the forward end of the plunger rod.

According to the invention in a further aspect, there is provided a method of assembling an auto-injector subassembly for use with an auto-injector, the method comprising: at least partially inserting a plunger assembly into a barrel of a syringe, the plunger assembly comprising a cartridge and a firing assembly, the firing assembly comprising a plunger rod and a drive portion and being axially moveable relative to the cartridge, such that a forward end of the plunger rod meets a bung located within the barrel; coupling the firing assembly to the cartridge at a coupling point along a length of the cartridge to fix an axial position of the drive portion relative to the cartridge; and moving at least part of the cartridge rearwards such that the coupling point and the forward end of the plunger rod move rearwards.

Optionally, moving at least part of the cartridge rearwards comprises extending the length of the cartridge in a rearward direction.

Optionally, the firing assembly further comprises firing assembly locking features, and coupling the firing assembly to the cartridge comprises engaging the firing assembly locking features with cartridge locking features on the cartridge.

Optionally, engaging the firing assembly locking features with the cartridge locking features comprises rotating at least one of the firing assembly and the cartridge with respect to the other of the firing assembly and the cartridge.

Optionally, the cartridge comprises a first portion and a second portion, and extending the cartridge rearwards comprises axially moving one of the first portion and the second portion relative to the other of the first portion and the second portion.

Optionally, one of the first and second portions of the cartridge comprises an angled surface configured to interact with a cartridge follower on the other of the first and second portions of the cartridge and configured to ride over the angled surface, and extending the cartridge rearwards comprises rotating the second portion relative to the first portion to cause the corresponding feature to ride over the angled surface.

Optionally, the method further comprises rotationally fixing the first portion relative to a housing of the auto-injector.

Optionally, the plunger assembly further comprises an actuator axially coupled to the cartridge, and the method further comprises inserting the cartridge into the auto-injector by applying an axial force to the actuator, preventing further forward movement of the cartridge, continuing to apply the axial force to the actuator while further forward movement of the cartridge is prevented such that the axial coupling between the actuator and the cartridge is overcome and a cartridge coupler of the actuator couples the firing assembly to the cartridge.

Optionally, the axial coupling comprises an angled surface on the actuator or the cartridge that is configured to interact with a locking follower on the other of the actuator or the cartridge, and continuing to apply the axial force to the actuator causes translation, at least partially, of the continued application of the axial force into a rotational force to couple the firing assembly to the cartridge.

Optionally, the, or a further, ramped surface is configured to interact with the locking follower, or a further follower, and moving at least part of the cartridge rearwards comprises causing the ramped surface to interact with the locking follower to rotate the second portion of the cartridge relative to the first portion of the cartridge.

According to the invention in a further aspect, there is provided a plunger assembly for use with an auto-injector, the plunger assembly comprising: a cartridge; and a firing assembly comprising a plunger rod and a drive portion, the firing assembly being axially moveable relative to the cartridge, wherein firing assembly is configured to couple to the cartridge at a coupling point along a length of the cartridge to fix an axial position of the drive portion relative to the cartridge to define an axial length of the plunger assembly.

Optionally, the firing assembly is telescopically received within the cartridge.

Optionally, the plunger assembly further comprises firing assembling locking features configured to engage with cartridge locking features for coupling the firing assembly to the cartridge.

Optionally, the firing assembly locking features are configured to engage with the cartridge locking features under relative rotation between the firing assembly and the cartridge.

Optionally, the plunger assembly further comprises an actuator axially coupled to the cartridge such that an axial force applied to the actuator inserts the cartridge into the auto injector, wherein the axial coupling is configured to be overcome on continued application of the axial force when forward movement of the cartridge is prevented, and wherein the actuator further comprises a cartridge coupler configured to couple the firing assembly to the cartridge on the continued application of the axial force.

Optionally, one of the actuator and cartridge comprises an angled surface that is configured to interact with a locking follower on the other of the actuator or the cartridge for translating, at least partially, the continued application of the axial force into a rotational force, and wherein the angled surface and the locking follower are configured to provide the axial coupling.

Optionally, the actuator comprises a sleeve configured to receive the cartridge therein.

According to the invention in a further aspect, there is provided a method of assembling an auto-injector subassembly for use with an auto-injector, the method comprising: at least partially inserting a plunger assembly into a barrel of a syringe, the plunger assembly comprising a cartridge and a firing assembly, the firing assembly comprising a plunger rod and a drive portion and being axially moveable relative to the cartridge, such that a forward end of the plunger rod meets a bung located within the barrel; coupling the firing assembly to the cartridge at a coupling point along a length of the cartridge to fix an axial position of the drive portion relative to the cartridge to define an axial length of the plunger assembly.

Optionally, the firing assembly further comprises firing assembly locking features, and coupling the firing assembly to the cartridge comprises engaging the firing assembly locking features with cartridge locking features on the cartridge.

Optionally, engaging the firing assembly locking features with the cartridge locking features comprises rotating at least one of the firing assembly and the cartridge with respect to the other of the firing assembly and the cartridge.

Optionally, the plunger assembly further comprises an actuator axially coupled to the cartridge, and the method further comprises inserting the cartridge into the auto-injector by applying an axial force to the actuator, preventing further forward movement of the cartridge, continuing to apply the axial force to the actuator while further forward movement of the cartridge is prevented such that the axial coupling between the actuator and the cartridge is overcome and a cartridge coupler of the actuator couples the firing assembly to the cartridge.

Optionally, the axial coupling comprises an angled surface on the actuator or the cartridge that is configured to interact with a locking follower on the other of the actuator or the cartridge, and continuing to apply the axial force to the actuator causes translation, at least partially, of the continued application of the axial force into a rotational force to couple the firing assembly to the cartridge.

According to the invention in a further aspect there is provided an auto-injector subassembly comprising a plunger assembly according to any of claims 20 to 26, and further comprising a housing configured to receive the plunger assembly.

Optionally, the auto-injector subassembly further comprises an axial end stop configured to prevent further forward movement of the plunger assembly.

Optionally, the housing is configured to receive a syringe, and further configured to position the syringe such that at least part of the syringe forms the axial end stop.

Optionally, the housing is configured to receive a syringe comprising a barrel and a bung located therein, and wherein the coupling point is located such that a forward end of the plunger rod contacts the bung.

According to the invention in a further aspect, there is provided an auto-injector comprising the plunger assembly of any of claims 1 to 12 and claims 20 to 26, and/or the auto-injector subassembly of any of claims 13 to 16 and claims 29 to 30.

### BRIEF DESCRIPTION OF THE DRAWINGS

FIG. 1a shows an isometric view of part of an exemplary plunger assembly;

FIG. 1b shows a side elevation of a first portion and part of a second portion of a cartridge of an exemplary plunger assembly;

FIG. 2 shows an isometric view of an exemplary plunger assembly;

FIGS. 3a and 3b show a partial section through an exemplary auto-injector subassembly; and

FIGS. 4a-4c show a partial side elevation of an exemplary plunger assembly at different stages of operation.

### DESCRIPTION

Typically ready-to-use auto-injectors may be provided to a user such that a forward end of a plunger rod is held in contact with the bung, and the bung, in turn, is held in contact with a substance, such as a medicament, contained within the barrel. The plunger rod and the bung may be held in position such that rearward movement away from the medicament is not possible in an assembled (or ready-to-use/pre-use) configuration.

The inventors have realised that such configurations do not permit expansion of a substance contained within the barrel of a syringe, forward of the bung. In one specific case, transporting such pre-filled syringes in low-pressure environments (such as at high altitude) may cause leakage of the medicament, since if the plunger rod and the bung are fixed in position, there is no volume into which the medicament can expand except out of a forward end of the syringe.

Generally disclosed herein are exemplary plunger assemblies for use with auto-injectors. Exemplary plunger assemblies may comprise a cartridge and a firing assembly comprising a plunger rod and a drive portion. In specific arrangements, prior to assembly of the auto-injector, the firing assembly is moveable axially with respect to the cartridge until a relative position of the firing assembly with respect to the cartridge is set, during assembly. After the relative position is set, the firing assembly can be considered to be coupled to the cartridge in that forward movement of the cartridge results in forward movement of the plunger rod. In exemplary arrangements, the coupling of the firing assembly to the cartridge fixes an axial position of the drive portion relative to the cartridge. In some exemplary arrangements, a syringe may be retained in a housing of an auto-injector. The plunger assembly may be inserted into the housing and the plunger rod is, in turn, inserted into the barrel of a syringe during assembly of the auto-injector.

A forward end of the plunger rod may contact the bung and because the firing assembly is axially moveable relative to the cartridge, forward movement of the plunger rod is held by the bung while the cartridge continues to move forwards until correctly seated or fixed within the housing. At this point, the firing assembly and/or the cartridge are configured to couple together, at least axially, at a coupling point and the plunger rod is of a length that it is in contact with the bung. The cartridge is configured such that the coupling point may be moved rearwards. Movement of the coupling point rearwards moves the plunger rod rearwards and thereby moves the forward end of the plunger rod away from the bung, creating a separation therebetween. In exemplary arrangements of the plunger assembly the length of the cartridge is increaseable. The length of the cartridge may be increased rearwards so that the coupling point moves rearwards.

When exemplary plunger assemblies are used with auto-injectors, a separation is defined between the bung and a forward end of the plunger rod when the auto-injector is in an assembled condition. The separation between the bung and the forward end of the plunger rod accommodates expansion of a medicament in a barrel of the pre-filled syringe, which prevents leaks.

Throughout the specification, the term “forward” refers to the end of the auto-injector from which the medicament is delivered. In other words, the forward end of the autoinjector is the end proximal to an injection site during use. The terms “rear” or “rearward” refer to the plunger end of the auto-injector or component thereof. In other words, the term “rearward” means distant or remote from the injection site during use. Other relative terms such as axial, longitudinal and the like are used to aid description of the device and need not be seen as limiting on the scope of the invention as claimed.

The term “cartridge” may encompass any sleeve, collar or other component capable of receiving the firing assembly. For example, a sleeve, collar or other component configured to telescopically receive the firing assembly such that the firing assembly is moveable therein.

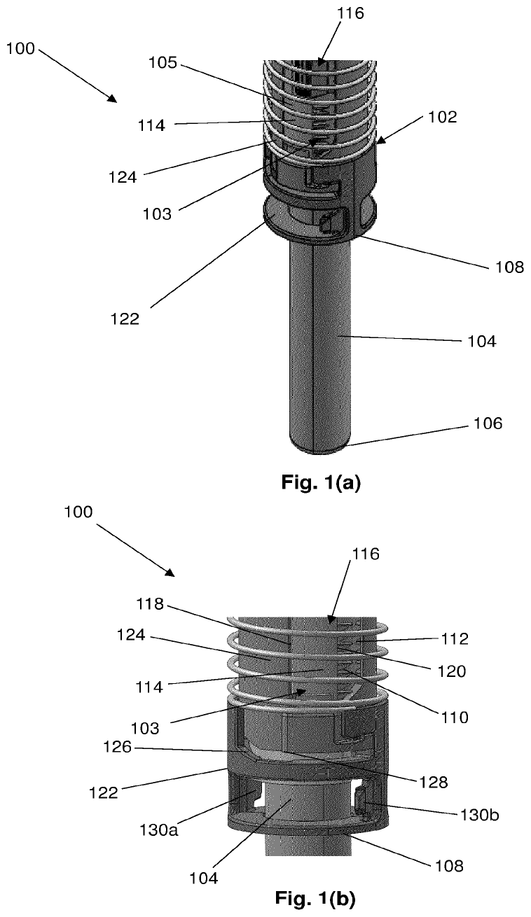

FIG. 1a shows an exemplary plunger assembly 100. The plunger assembly 100 comprises a cartridge 102 and a firing assembly 103 comprising a plunger rod 104 and a drive portion 105. The firing assembly 103 is axially moveable relative to the cartridge 102. As such, the plunger rod 104 is axially moveable relative to the cartridge 102.

The plunger rod 104 may be coupled to the drive portion 105 such that axial movement of the plunger rod 104 causes axial movement of the drive portion 105 and vice versa. As such, relative movement of the firing assembly 103 with respect to the cartridge 102 also encompasses relative movement of the plunger rod 104 with respect to the cartridge 102.

In exemplary arrangements, the firing assembly 103 may further comprise a delivery driver disposed between the drive portion 105 and the plunger rod 104. The delivery driver may bias the plunger rod 104 forwards with respect to the drive portion 105. The plunger rod 104 may be moveable relative to the drive portion 105 on a delivery stroke, under the bias of the delivery driver, on decoupling of the plunger rod 104 and the drive portion 105.

In the example shown, the firing assembly 103 is received within the cartridge 102. A forward end 106 of the plunger rod 104 extends from a forward end 108 of the cartridge 102. The plunger rod 104 is telescopically received within the cartridge 102 such that 25 the extent that the forward end 106 of the plunger rod 104 extends from the forward end 108 of the cartridge 102 may be varied.

The skilled person will appreciate that alternative arrangements that allow for relative axial movement between the firing assembly 103 and the cartridge 102 may be utilised.

For example, in alternative arrangements, the cartridge 102 and the firing assembly 103 may be configured to slide alongside each other. The skilled person will be able to envisage further configurations.

The firing assembly 103 may comprise firing assembly locking features 110 (best shown in FIG. 1 b). The firing assembly locking features 110 may be configured to engage with cartridge locking features 112 to couple the plunger rod 104 to the cartridge 102.

The firing assembly locking features 110 may be configured to engage with the cartridge locking features 112 at a coupling point. In the exemplary plunger assembly 100, the firing assembly 103 may be capable of coupling to the cartridge 102 at a plurality of positions along the length of the cartridge 102. As such, the term “coupling point” encompasses the one of the plurality of positions along the length of the cartridge 102 at which the firing assembly 103 couples to the cartridge 102. The axial position of the drive portion 105 and/or the plunger rod 104 relative to the cartridge 102 may be fixed when the firing assembly 103 and the cartridge 102 are coupled, e.g. when the firing assembly locking features 110 are engaged with cartridge locking features 112.

In the exemplary plunger assembly 100 the drive portion 105 comprises the firing assembly locking features. In such arrangements, engagement of the firing assembly locking features 110 with the cartridge locking features 112 may fix an axial position of the drive portion 105 relative to the cartridge 102. As described above, in arrangements in which the plunger rod 104 is coupled to the drive portion 1 05, fixing the axial position of the drive portion 105 relative to the cartridge 102 fixes the axial position of the plunger rod 104 relative to the cartridge 102.

In the exemplary plunger assembly 100 of FIG. 1 a and 1 b, the firing assembly locking features 110 and the cartridge locking features 112 comprise teeth. The teeth of the firing assembly locking features 110 and the teeth of the cartridge locking features 112 may be configured to mesh to couple the firing assembly 103 and the cartridge 106. In the exemplary arrangement shown in FIGS. 1a and 1b, the teeth of the firing assembly 103 and the teeth of the cartridge 102 may be rearwardly angled. This ensures that the teeth are able to mesh regardless of the axial position of the firing assembly 103 with respect to the cartridge 102 prior to coupling. That is, if the teeth of the firing assembly locking features 110 and the teeth of the cartridge locking features 112 are misaligned prior to engagement then the rearwardly angled teeth serve to guide the respective teeth into alignment.

The skilled person will appreciate that alternative firing assembly locking features 110 and cartridge locking features 112 may be utilised. For example, a lug and recess arrangement, clips, a magnetic arrangement, and/or a ratchet arrangement. The skilled person will be able to envisage further configurations. The skilled person will also appreciate that although the exemplary locking features 110 and 112 shown in FIGS. 1a and 1b provide a plurality of discrete positions along the length of the cartridge 102 to which the firing assembly 103 may couple, continuous coupling arrangements may be provided such that the firing assembly 103 may couple to the cartridge 102 at substantially any point along the length of the cartridge 102.

In the exemplary arrangement of FIG. 1 a and 1 b, the cartridge 102 and the firing assembly 103 each comprise a linear track of teeth extending along at least part of a length of the cartridge 102 and a length of the firing assembly 103 respectively. In the arrangement of FIG. 1 a and 1 b, the linear track of teeth extends along at least part of a length of the drive portion 105 of the firing assembly 103. The skilled person will appreciate that other teeth arrangements are possible, provided that the teeth of the firing assembly 103 are able to mesh with the teeth of the cartridge 102 at a plurality of points along the length of the cartridge 102. The pitch of the teeth of the linear track of the cartridge 102 and the firing assembly 103 may be equal. In the exemplary plunger assembly 100, the length of the linear track of teeth of the cartridge 102 may be greater than the length of the linear track of teeth of the firing assembly 103.

The skilled person will appreciate that the number of teeth and the pitch of the teeth of may be varied depending on the number of potential coupling points desired along the length of the cartridge 102. As such, altering the number of teeth and/or the pitch of the teeth, will allow the number of potential coupling points to be varied.

The firing assembly 103 may comprise a projection 114. In the exemplary arrangement shown in FIG. 1 a and 1 b, the drive portion 105 comprises the projection 114. The projection 114 may be configured to interact with the cartridge 102 to prevent relative rotation between the firing assembly 103 and the cartridge 102 in a first direction. In the exemplary arrangement of FIG. 1 a and 1 b, the projection 114 is received within a track 116 of the cartridge 102. An edge of the track 116 may comprise a stop surface 118. Relative rotation between the firing assembly 103 and the cartridge 102 in the first direction may be prevented when the projection 114 abuts the stop surface 118 of the cartridge 102.

The track 116 of the cartridge 102 may be an axial (or vertical) track arranged substantially in parallel to the longitudinal axis of the cartridge 102. The track 116 of the cartridge 102 may act as a guide for the axial movement of the firing assembly 103 relative to the cartridge 102. In the exemplary arrangement of FIG. 1 a and 1 b, the projection 114 may be configured to ride along the stop surface 118 of the track 116 when the firing assembly 103 is moved axially within the cartridge 102.

In the exemplary firing assembly 103 shown in FIG. 1 a and 1 b, the firing assembly locking features 110 may be arranged such that relative rotation between the firing assembly and the cartridge 102 engages the firing assembly locking features 110 and the cartridge locking features 112. The relative rotation to engage the firing assembly locking features 110 and the cartridge locking features 112 may be in a second direction, opposite to the first direction.

The firing assembly 103 may further comprise a coupling surface 120. In the exemplary arrangement of FIG. 1 a and 1 b, the projection 114 comprises the coupling surface 120, however in alternative arrangements, the coupling surface 120 may be separate from the projection 114. The coupling surface 120 may be configured to interact with the cartridge 102 to rotationally couple the firing assembly 103 to the cartridge 102. In the examples shown in FIG. 1 a and 1 b, the drive portion 105 comprises the coupling surface 120.

The linear track of teeth that form at least part of the firing assembly locking features 110 in the exemplary arrangement of FIG. 1 a and 1 b, may extend from the projection 114.

The teeth of the firing assembly 103 may extend from the coupling surface 120 of the projection 114 which is opposed to the surface of the projection 114 configured to interact with the stop surface 118 of the track 116. As such, relative rotation between the firing assembly 103 and the cartridge 102 in the second direction may cause the corresponding teeth of the firing assembly 103 and the cartridge 102 to mesh.

In general, at least part of the cartridge 102 is moveable rearwards in order to move the coupling point rearwards after coupling of the firing assembly 103 with the cartridge 102. In the exemplary cartridge 102 a length of the cartridge may be increased or extended rearwards.

The exemplary cartridge 102 of FIG. 1 a and 1 b comprises a first portion 122 and a second portion 124. At least one of the first portion 122 and the second portion 124 may be axially moveable relative to the other of the first portion 122 and the second portion 124. The relative axial movement between the first portion 122 and the second portion 124 may cause the extension of the cartridge 102 rearwards. In the exemplary arrangement of FIG. 1 a and 1 b, relative rotation between the first portion 122 and the 5 second portion 124 may cause the relative axial movement therebetween.

At least one of the first portion 122 and the second portion 124 may comprise an angled surface 126 configured to interact with a cartridge follower 128 on the other of the first portion 122 and the second portion 124. In the exemplary arrangement of FIGS. 1a and 1b, the first portion 122 is forward of the second portion 124, and comprises the angled surface 126. The second portion 124 comprises the cartridge follower 128. In alternative arrangements, the skilled person will appreciate that the first portion 122 may comprise the cartridge follower 128 and the second portion 124 may comprise the angled surface 126. The cartridge follower 128 may be configured to ride over the angled surface 126 on relative rotation between the first portion 122 and the second portion 124.

The skilled person will appreciated that the phrase “ride over” encompasses travel of the corresponding feature along the angled surface, and is not intended to impose a positional limitation. That is, the cartridge follower 128 need not be located “over” the ramped surface. In the arrangement shown in FIG. 1 a and 1 b, the cartridge follower 128 comprises a projection. In alternative arrangements, the cartridge follower 128 may comprise a peg or a lug, a corresponding angled surface or any other feature capable of riding over the angled surface 126 on relative rotation between the first portion 122 and the second portion 124. In alternative arrangements the angled surface 126 and the cartridge follower 128 may comprise corresponding threads.

The angled surface 126 is angled rearwards. Therefore, as the cartridge follower 128 rides over the angled surface 126, the second portion 124 is moved axially rearwards relative to the first portion 122. As such, the angled surface 126 translates rotational force into axial force on relative rotation between the first portion 122 and the second portion 124. The skilled person will appreciate that in alternative arrangements, other methods of causing relative axial movement between the first portion 122 and the second portion 124 may be utilised. For example, a biasing member may be configured to be released to bias the first and second portions away from one another when released from a primed state. The skilled person will be able to envisage other arrangements. In addition, the skilled person will understand that the cartridge 102 itself may be moved rearwards after coupling of the cartridge 102 to the plunger rod 104.

The cartridge 102 may comprise a rotational lock. The rotational lock may be configured to rotationally fix at least one of the first portion 122 and the second portion 124 relative to a housing of the auto-injector into which the exemplary plunger assembly 100 may be inserted. In the arrangement shown in FIG. 1 a and 1 b, the rotational lock is configured to rotationally fix the first (or forward) portion 122 relative to a housing (described in more detail below). The rotational lock may comprise arms 130a, 130b.

The arms 130a, 130b may extend radially outwardly from the first portion 122. The arms 130a, 130b may be configured to interact with a corresponding feature on the housing of the auto-injector to prevent relative rotation between the housing and the first portion 122. For example, the arms 130a, 130b may be configured to interact with a projection, such as a track, located on, or coupled to, the housing. The skilled person will appreciate that alternative rotational locks may be utilised, for example, the housing may comprise a keyed aperture within which the first portion 122 is received. The skilled person will be able to envisage alternative possibilities. In further alternative arrangements the rotational lock may be configured to rotationally fix the second (rearward) portion 124 with respect to the housing instead of the first portion 122.

In the exemplary plunger assembly 100, the second (or rearward) portion 124 may comprise the cartridge locking features 112, the track 116 and the stop surface 118. As such, in the exemplary plunger assembly 100, the firing assembly 103 may be configured to couple to the second portion 124 and the coupling point is therefore on the second portion 124.

The track 116 extends along substantially the entire length of the second portion 124.

As described above, in the exemplary arrangement shown in Figures a and 1b, the cartridge locking features 112 comprise teeth. The teeth may be located on a radially inner surface of the second portion 124. In the exemplary arrangement of FIGS. 1a and 1b, the teeth are arranged proximal to an edge of the track 116. The teeth may be arranged on the edge of the track 116 opposite to the edge of the track 116 that comprises the stop surface 118. The linear track of teeth of the cartridge locking features 112 may extend substantially over the entire length of the second portion 124.

In alternative arrangements, a housing of an auto-injector into which the plunger assembly 100 is to be inserted may comprise one or more of the features of the first portion 122. For example, the first portion 122 may be integral with the housing of the auto-injector and configured to interact with the second portion 124 on insertion of the cartridge 102 within the housing. In other arrangements, the housing may comprise alternative features configured to interact with the cartridge 102 to move the coupling point rearwards after coupling of the firing assembly 103 with the cartridge 102. For example, one of the housing and the cartridge 102 may comprise a surface configured to interact with a corresponding feature on the other of the housing and the cartridge 102 at a point during insertion of the plunger assembly 102 to cause rearward movement of the cartridge 102 within the housing. The surface may be a ramped surface (for example, as described above) and the interaction may cause relative rotation between the cartridge 102 and the housing (and as such the firing assembly 103) to move the coupling point rearwards. In such arrangements, the cartridge 102 may comprise one of the first portion 122 and the second portion 124.

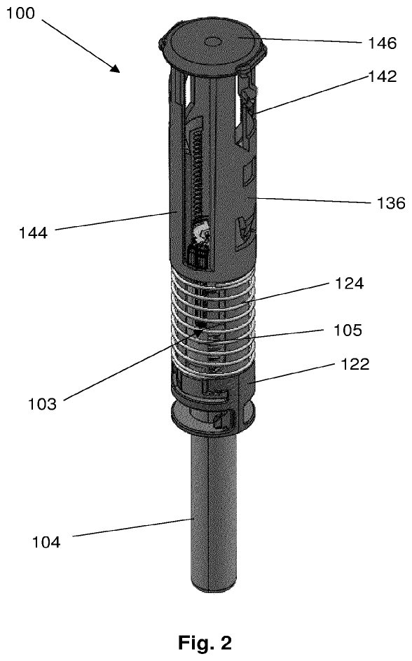

The plunger assembly 100 may further comprise an actuator 136 (shown in FIG. 2).

The actuator 136 may be axially moveable relative to the cartridge 102 upon application of an axial force to the actuator 136.

The actuator 136 may be configured to cause relative rotation between the first portion 122 and the second portion 124 on application of the axial force to the actuator 136. The axial force may be a forward force. For example, the axial force may be applied during insertion of the plunger assembly 100 into a housing of an auto-injector device.

As discussed above, relative rotation between the first portion 122 and the second portion 124 may cause the firing cartridge locking features 110 and the cartridge locking features 112 to engage. In exemplary arrangements relative rotation between the first portion 122 and the second portion 124 may additionally, or alternatively, cause relative axial movement between the first portion 122 and the second portion 124.

In the exemplary arrangement of FIG. 2, the actuator 136 comprises a cartridge coupler configured to cause the cartridge 102 to couple to the firing assembly 103 on application of an axial force to the actuator 136. The cartridge coupler may comprise a ramped surface 142 configured to interact with a locking follower (not visible in FIG. 2) located on the second portion 124 of the cartridge 102. The skilled person will appreciate that in alternative arrangements, the second portion 124 of the cartridge 102 may comprise the ramped surface and the actuator may comprise the locking follower. In the exemplary arrangement of FIG. 2, the locking follower comprises a corresponding ramped surface, however the skilled person will appreciate that in alternative arrangements, the locking follower may comprise a lug, projection or peg, or any other component capable of riding over the ramped surface. The ramped surface 142 may be rearwardly extending. The locking follower may be configured to be located at a forwardmost position of the ramped surface 142 in an initial configuration of the plunger assembly 100.

In the exemplary arrangement of FIG. 2, the actuator 136 comprises a sleeve 144.

The actuator may further comprise an end cap 146. The end cap 146 may be configured to abut a rearward face of the housing of the auto-injector device when the plunger assembly 100 is fully inserted therein.

In the exemplary arrangement of FIG. 2, the sleeve 144 extends from the end cap 146.

The sleeve 144 may be configured to receive the cartridge 102. In the exemplary actuator 136, the sleeve 144 is configured to receive the second portion 124 of the cartridge 102.

In such arrangements, the sleeve 144 may comprise the ramped surface 142 (or in arrangements in which the second portion 124 comprises the ramped surface 142, the sleeve 144 may comprise the locking follower).

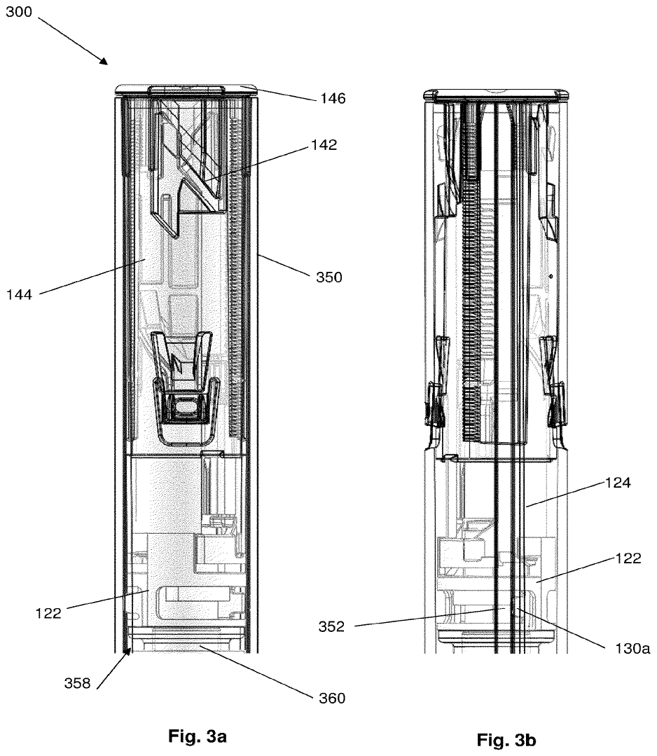

FIGS. 3a and 3b show an auto-injector subassembly 300. The auto-injector subassembly 300 comprises the plunger assembly 100 of FIG. 1 a, 1 b and 2, and a housing 350. The plunger assembly 100 may be configured for insertion into the housing 350.

The housing 350 may comprise rotational engagement features 352. The rotational engagement features 352 may be configured to engage the rotational lock of the cartridge 102. The rotational engagement features 352 of the housing 350 may be configured to engage the rotational lock of the first portion 122 of the cartridge 102 to prevent rotation of the first portion 122 relative to the housing 350.

In the exemplary arrangement of FIGS. 2, 3a and 3b, the rotational engagement features 352 comprise an inwardly extending projection. The skilled person will appreciate that alternative rotational engagement features 352 may be used to prevent rotation of the first portion 122 relative to the housing 350. For example, in alternative arrangements, the housing may comprise a keyed aperture configured to receive the first portion, or a high friction material disposed on an inner face of the housing configured to engage a face of the first portion to resist rotation thereof. The skilled person will be able to envisage further arrangements.

The projection may extend along at least a portion of the length of the housing 350 to form a track. In such arrangements, the rotational lock of the first portion 122 of the cartridge 102 may engage the rotational engagement features 352 on insertion of the plunger assembly 100 into the housing 350. In alternative arrangements, the rotational engagement feature 352 may comprise a discrete projection located to engage the rotational lock of the cartridge 102 when the plunger assembly 100 is fully received within the housing 350.

In the exemplary arrangement of FIGS. 3a and 3b, the projection may be configured to interact with one of the arms 130a, 130b of the first portion 122 of the cartridge 102. In the exemplary arrangement of FIGS. 3a and 3b, the rotational engagement features 352 comprise two inwardly extending projections. The two inwardly extending projections may be opposed and configured to engage corresponding arms 130a, 130b of the first portion 122. Rotation of the first portion 122 relative to the housing 350 may be prevented in a first direction by an engagement between a first projection and a first arm 130a, and rotation of the first portion 122 in a second direction, opposite to the first direction, may be prevented by engagement between a second projection and a second arm 130b.

The auto-injector subassembly 300 may comprise an axial end stop 358. The axial end stop 358 may be configured to prevent forward movement of the plunger assembly 100 when the plunger assembly 100 is engaged with the axial end stop 358. The plunger assembly 100 may be configured to engage the axial end stop 358 during insertion of the plunger assembly 100 within the housing 350.

The housing 350 may be configured to receive a syringe 360. In the exemplary arrangement shown in FIGS. 3a and 3b, a portion of the syringe 360 forms the axial end stop 358. The skilled person will appreciate that in alternative arrangements, alternative axial end stop 358 may be utilised. For example, the axial end stop 358 may comprise an inwardly extending projection or lip located on the housing and configured to engage a corresponding feature on the plunger assembly 100 to prevent forward movement within the housing 350.

A forward end of the cartridge 102 may be configured to abut the syringe 360 during insertion of the plunger assembly 100 within the housing 350. In the exemplary arrangement of FIGS. 3a and 3b, the first portion 122 may be configured to abut the syringe 360. In alternative arrangements, the first portion 122 may be configured to abut a component disposed between the syringe 360 and the first portion 122. When the first portion 122 and the syringe 360 are abutting, further forward movement of the plunger assembly 100 is prevented.

Operation of the plunger assembly 100 is described below with reference to FIGS. 2, 3a and 3b, and 4a-4c.

FIG. 2 shows the plunger assembly 100 in an initial position. In the initial position, the firing assembly 103 and the cartridge 102 are decoupled, and the firing assembly 103 is axially moveable with respect to the cartridge 102. In exemplary arrangements, in the initial position, the actuator 136 and the cartridge 102 may be axially coupled, such that axial movement of one of the actuator 136 and the cartridge 102 causes axial movement of the other of the actuator 136 and the cartridge 102. An axial force may be applied to the actuator 136 to insert the cartridge 102 into an auto-injector. In the examples shown in FIGS. 3a and 3b, an axial force is applied to the actuator 136 to insert the cartridge 102 into the housing 350.

The plunger assembly 100 may be inserted into a barrel of the syringe 360. In the exemplary arrangement shown in FIGS. 3a and 3b, inserting the plunger assembly 100 into the barrel of the syringe 360 comprises inserting the plunger assembly 100 into the housing 350.

Insertion of the plunger assembly 100 within the housing 350 may engage the rotational lock of the cartridge 102 with the rotational engagement features 352 of the housing 350.

In the exemplary arrangement shown in FIGS. 3a and 3b, insertion of the plunger assembly 100 into the housing 350 brings the arms 130a, 130b of the first portion 122 of the cartridge 102 into engagement with the corresponding projections on the housing 350. Engagement of the arms 130a, 130b with the corresponding projections prevents rotation of the first portion 122 relative to the housing 350.

The plunger assembly 100 may be moved forwards within the housing 350. In exemplary arrangements, the plunger assembly 100 may be moved forwards by applying an axial force to the actuator 136. Since the actuator 136 is axially coupled to the cartridge 102, application of the axial force to the actuator 136 causes the cartridge 102 and the firing assembly 103 to move forwards within the housing 350. At a point during the forward movement of the plunger assembly 100, the forward end 106 of the plunger rod 104 may contact a bung located within the barrel of the syringe 360. The skilled person will appreciate that the syringe 360 may be a pre-filled syringe and the point at which the plunger rod 104 contacts the bung will depend on the fill volume of the pre-filled syringe.

Once the plunger rod 104 contacts the bung, further forward movement of the firing assembly 103 is prevented. As such, further forward force applied to the plunger assembly 100 causes relative axial movement between the plunger rod 104 and the cartridge 102.

In the exemplary arrangement of FIGS. 3a and 3b, since the actuator 136 and the cartridge are coupled, further forward movement of the actuator under the axial force causes further forward movement of the cartridge 102. The cartridge 102 therefore continues to move forwards within the housing 350, while the firing assembly 103 remains in substantially the same axial position relative to the housing 350. As such, more of the length of the plunger rod 104 is received within the cartridge 102 and the extent to which the forward end 106 of the plunger rod 104 extends past the forward end 108 of the cartridge 102 begins to reduce.

The cartridge 102 may be moved further forwards until the plunger assembly 100 engages the axial end stop 358. Further forward movement of the cartridge 102 is prevented when the plunger assembly 100 engages the axial end stop 358. In the exemplary arrangement shown in FIGS. 3a and 3b, the syringe 360 forms the axial end stop 358, and the plunger assembly 100 is moved further forwards until the cartridge 102 is brought into engagement with the syringe 360. In exemplary arrangements, the plunger assembly 100 may be moved forwards within the housing 350 until the cartridge 102 abuts the syringe 360, for example, until the first portion 122 of the cartridge 102 abuts the syringe 360.

Continued application of the axial force on the actuator 136 when forward movement of the cartridge 102 is prevented may overcome the axial coupling between the cartridge 102 and the actuator 136. Further application of axial force to the actuator 136 after the axial coupling is overcome may cause the cartridge coupler of the actuator 136 to couple the firing assembly 103 to the cartridge 102. The axial coupling, or the features resulting in the axial coupling, may comprise the angled surface 142 of the actuator 136 and the locking follower on the cartridge 102. In such arrangements, when the cartridge is free to move forwards, application of axial force to the actuator 136 may cause corresponding axial movement of the cartridge. In such arrangements, the locking follower may not travel along the angled surface 142 when the cartridge 102 is free to move forwards.

When forward movement of the cartridge 102 is prevented however, continued application of axial force to the actuator may cause the locking follower to travel along the angled surface 142 to translate, at least partially, the continued application of the axial force into a rotational force. That is, the axial coupling may be overcome on continued application of the axial force to the actuator when forward movement of the cartridge is prevented.

The skilled person will appreciate that the coupling point at which the firing assembly 103 couples to the cartridge 102 may depend on the location of the bung within the barrel of the syringe. That is, the lower the fill volume of the syringe, the further forward the bung will be within the barrel of the syringe, and therefore the further forward the coupling point.

In the exemplary arrangement of FIGS. 3a and 3b, relative rotation between the cartridge 102 and the firing assembly 103 causes the firing assembly 103 and the cartridge 102 to couple. When the firing assembly 103 is coupled to the cartridge 102, the axial position of the firing assembly 103 relative to the cartridge 102 is fixed. In the examples shown in FIGS. 3a and 3b, the axial position of the drive portion 105 is fixed relative to the cartridge 102. The axial position of the firing assembly 103 (or specifically the drive portion 105) relative to the cartridge 102 may be fixed such that the forward end 106 of the plunger rod 104 is in contact with the bung.

In the exemplary arrangement of FIGS. 3a and 3b, the relative rotation between the cartridge 102 and the firing assembly 103 to couple the firing assembly 103 and the cartridge 102 is caused by the interaction between the ramped surface 142 of the actuator and the locking follower on the second portion 124 of the cartridge 102. Since the cartridge 102 is unable to move further forwards, further axial force applied to the actuator 136 overcomes the axial coupling between the actuator 136 and the cartridge 102. Overcoming the axial coupling causes the locking follower on the second portion 124 of the cartridge 102 to ride over the ramped surface 142 of the actuator 136. This decouples the cartridge 102 from the actuator 136 and causes the second portion 124 to rotate relative to the actuator 136. This, in turn, causes relative rotation between the second portion 124 and the firing assembly 103. As such, the cartridge locking features 112 are brought into engagement with the firing assembly locking features 110. In the arrangement shown in FIGS. 3a and 3b, bringing the cartridge locking features 112 and the firing assembly locking features 110 into engagement comprises causing the corresponding teeth to mesh.

Since the rotational lock of the first portion 122 of the cartridge 1 02 is engaged with the rotational engagement features 352, the second portion 124 also rotates relative to the first portion 122.

In alternative arrangements, the relative rotation between the cartridge 102 and the firing assembly 103 may be caused by rotating the housing 350 relative to the cartridge 102.

In such arrangements, the cartridge 102 may comprise a coupling feature configured to couple the cartridge 102 to the housing 350 on insertion of the cartridge 102 therein. In such arrangements, rotation of the housing 350 may cause engagement of the coupling feature on the cartridge and a corresponding coupling feature on the housing 350, such that rotation of the housing 350 causes rotation of the cartridge 102 relative to the firing assembly 103. The relative rotation between the firing assembly 103 and the cartridge 102 may engage the firing assembly locking features 110 with the cartridge locking features 112. The skilled person will appreciate such arrangements may not comprise an actuator 136. In this way, the cartridge 102 and the firing assembly 103 may be inserted into the housing 350 axially, and the housing rotated to couple the firing assembly 103 to the cartridge 102 and/or move the coupling point rearwards.

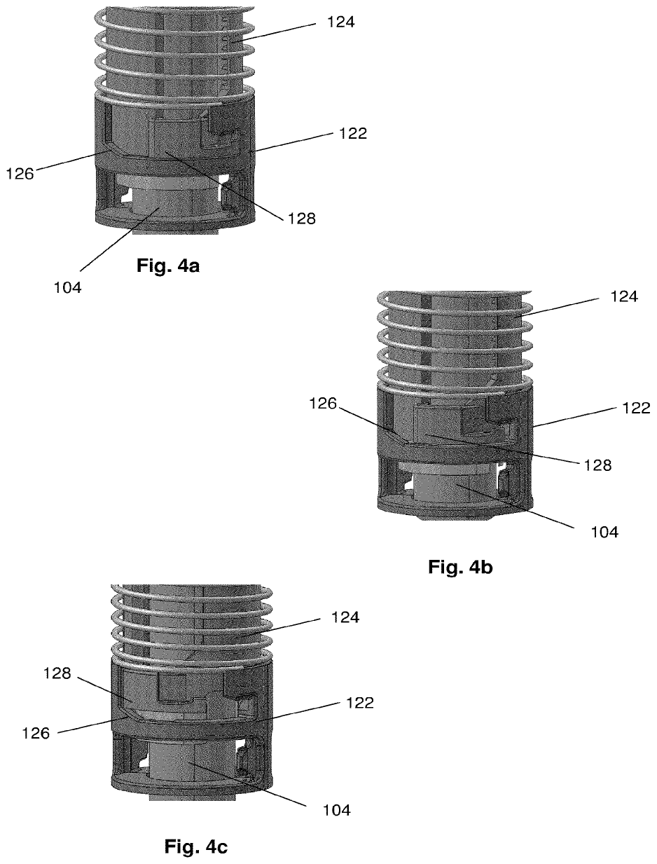

FIGS. 4a-4c show the relative positions of the first portion 122 and the second portion 124 at various points during the rotation of the second portion 124.

FIG. 4a shows the relative positions of the first portion 122 and the second portion 124 before the cartridge 102 and the firing assembly 103 couple (and as such, before the second portion 124 begins to rotate with respect to the firing assembly 103 and the first portion 122).

Rotation of the second portion 124 relative to the firing assembly 103 to couple the cartridge 102 and the firing assembly 103, causes the cartridge follower 128 of the second portion 124 to be rotated towards the angled surface 126 of the first portion 122.

FIG. 4b shows the relative positions of the first portion 122 and the second portion 124 at the point at which the firing assembly 103 and the cartridge 102 have coupled together.

That is, at the point at which the firing assembly locking features 110 and the cartridge locking features 112 have engaged.

Further rotation of the second portion 124 relative to the first portion 122 after the firing assembly has coupled to the cartridge 102 causes the cartridge follower 128 of the second portion 124 to ride over the angled surface 126 of the first portion 122. This causes the second portion 124 to move rearwards with respect to the first portion 122.

Since the coupling point between the firing assembly and the cartridge is on the second portion 124, the rearward movement of the second portion 124 also causes rearward movement of the coupling point and therefore plunger rod 104. As such, the plunger rod 104 is moved rearwardly with respect to the bung to define a separation therebetween. FIG. 4c shows the relative positions of the first portion 122 and the second portion 124 when the cartridge follower 128 of the second portion 124 has ridden over the angled surface 126 of the first portion 122, and the second portion 124 has moved axially rearwards with respect to the first portion 122.

The skilled person will appreciate that utilising the plunger assembly 100 described above allows a consistent separation between the plunger rod 104 and the bung in the syringe 360 to be defined, regardless of the fill volume of the syringe 360. This is because, the plunger rod 104 is firstly brought into contact with the bung, then coupled to the cartridge, and then the cartridge is moved axially rearwards by a predefined distance. As such, the plunger assembly 100 may be utilised with syringes of a variety of different fill volumes.

It is noted that many of the features of the exemplary plunger assembly described above and shown in the drawings may be included in other exemplary apparatus. As such, the different drawings are not necessarily to be considered as separate embodiments and features from one drawing may be transferred to an apparatus in another drawing. The skilled person will be able to envisage other plunger assemblies and features thereof without departing from the scope of the appended claims.

What is claimed is:

1. A plunger assembly for use with an auto-injector, the plunger assembly comprising:
a cartridge; and
a firing assembly comprising a plunger rod and a drive portion, the firing assembly being axially moveable relative to the cartridge,
wherein the firing assembly is configured to couple to the cartridge at a coupling point along a length of the cartridge to fix an axial position of the drive portion relative to the cartridge to define an axial length of the plunger assembly.

2. The plunger assembly of claim 1, wherein the firing assembly is telescopically received within the cartridge.

3. The plunger assembly of claim 1, further comprising firing assembly locking features configured to engage with cartridge locking features for coupling the firing assembly to the cartridge.

4. The plunger assembly of claim 3, wherein the firing assembly locking features are configured to engage with the cartridge locking features under relative rotation between the firing assembly and the cartridge.

5. The plunger assembly of claim 1, further comprising an actuator coupled to the cartridge via an axial coupling such that an axial force applied to the actuator inserts the cartridge into the auto-injector, wherein:
the axial coupling is configured to be overcome on continued application of the axial force when forward movement of the cartridge is prevented; and
the actuator further comprises a cartridge coupler configured to couple the firing assembly to the cartridge on the continued application of the axial force.

6. The plunger assembly of claim 5, wherein one of the actuator or cartridge comprises an angled surface that is configured to interact with a locking follower on the other of the actuator or the cartridge for translating, at least partially, the continued application of the axial force into a rotational force, and wherein the angled surface and the locking follower are configured to provide the axial coupling.

7. The plunger assembly of claim 5, wherein the actuator comprises a sleeve configured to receive the cartridge therein.

8. An auto-injector subassembly comprising the plunger assembly of claim 1, and further comprising a housing configured to receive the plunger assembly.

9. The auto-injector subassembly of claim 8, comprising an axial end stop configured to prevent further forward movement of the plunger assembly.

10. The auto-injector subassembly of claim 9, wherein the housing is configured to:
receive a syringe; and
position the syringe such that at least part of the syringe forms the axial end stop.

11. The auto-injector subassembly of claim 8, wherein the housing is configured to receive a syringe comprising a barrel and a bung located therein, and wherein the coupling point is located such that a forward end of the plunger rod contacts the bung.

12. An auto-injector comprising the plunger assembly of claims 1.

13. An auto-injector comprising the auto-injector subassembly of claim 8.

14. An auto-injector sub-assembly comprising a plunger assembly and a housing, the plunger assembly comprising:
a cartridge insertable within the housing; and
a firing assembly comprising a plunger rod and a drive portion, the firing assembly being axially moveable relative to the cartridge, wherein:
the firing assembly is configured to couple to the cartridge at a coupling point along a length of the cartridge to fix an axial position of the drive portion relative to the cartridge to define an axial length of the plunger assembly; and
the cartridge is configured to couple to the housing at a point during insertion therein.

15. The auto-injector sub-assembly of claim 14, wherein the firing assembly is telescopically received within the cartridge.

16. The auto-injector sub-assembly of claim 14, wherein the plunger assembly further comprises firing assembly locking features configured to engage with cartridge locking features for coupling the firing assembly to the cartridge.

17. The auto-injector sub-assembly of claim 14, wherein:
the plunger assembly further comprises an actuator coupled to the cartridge via an axial coupling such that an axial force applied to the actuator inserts the cartridge into the housing;
the axial coupling is configured to be overcome on continued application of the axial force when forward movement of the cartridge is prevented; and
the actuator further comprises a cartridge coupler configured to couple the firing assembly to the cartridge on the continued application of the axial force.

18. A method of assembling an auto-injector subassembly for use with an auto-injector, the method comprising:
at least partially inserting a plunger assembly into a barrel of a syringe, the plunger assembly comprising a cartridge and a firing assembly, the firing assembly comprising a plunger rod and a drive portion and being axially moveable relative to the cartridge, such that a forward end of the plunger rod meets a bung located within the barrel; and
coupling the firing assembly to the cartridge at a coupling point along a length of the cartridge to fix an axial position of the drive portion relative to the cartridge to define an axial length of the plunger assembly.

19. The method of claim 18, wherein the coupling the firing assembly to the cartridge comprises engaging firing assembly locking features with cartridge locking features.

20. The method of claim 18, wherein the plunger assembly further comprises an actuator coupled to the cartridge via an axial coupling, wherein the method further comprises:
applying an axial force to the actuator to insert the cartridge into a housing of the auto-injector subassembly;
overcoming the axial coupling on continued application of the axial force when forward movement of the cartridge is prevented; and
continuing application of the axial force to cause the coupling of the firing assembly to the cartridge at the coupling point along the length of the cartridge.
